(19) United States
(12) Patent Application Publication
(10) Pub. No.: US 2008/0184996 A1
(43) Pub. Date: Aug. 7, 2008

(54) BANDANA FOR A BREATHING FILTER ELEMENT

(76) Inventor: Juancarlos Colorado, Petaluma, CA (US)

Correspondence Address: STAINBROOK & STAINBROOK, LLP, 412 AVIATION BOULEVARD, SUITE H, SANTA ROSA, CA, 95403, US

(21) Appl. No.: 12/025,664

(22) Filed: Feb. 4, 2008

Related U.S. Application Data
(60) Provisional application No. 60/888,031, filed on Feb. 2, 2007.

(51) Int. Cl. A62B 7/10; A61B 19/00; A42B 5/00

(52) U.S. Cl. 128/201.25; 128/206.12; 128/898; 2/207

(57) ABSTRACT

A bandana for holding a particulate breathing filter over the mouth and nose of a wearer which includes a fabric panel with an outer surface, an inner surface, an upper edge, and at least first and second sides depending downwardly from the upper edge. The upper edge includes an upper elastic strap with first and second ends extending beyond the panel material sufficiently to tie or fasten the bandana around the head of a wearer. Attachment apparatus is disposed on the inner surface of the fabric panel so that a particulate air filter pouch having complementary attachment apparatus can be attached to the bandana.

### CROSS REFERENCES TO RELATED APPLICATIONS

The present application claims the benefit of U.S. Provisional Patent Application Ser. No. 60/888,031, filed Feb. 2, 2007 (Feb. 2, 2007).

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

1. Field of the Invention

The present invention relates generally to breathing filter apparatus, and more particularly to a bandana adapted for retaining and positioning a filter element over the mouth and nose of a wearer, and further adapted for rapid removal and replacement of such filter element.

2. Discussion of Related Art Including Information Disclosed under 37 CFR §§1.97, 1.98

Smoke inhalation is a principal danger to persons trapped in burning structures. It is a major cause of death and injury from fire. Filtering systems and breathing apparatus have been devised either to filter particulate smoke and toxic gas or to provide a respiratory barrier from the smoke and gas along with a fresh supply of oxygen.

A relatively inexpensive solution to filtering toxic gas is shown in U.S. Pat. No. 6,338,340, to Finch et al, which discloses a filter mask for use in smoke and toxic gas filled environments, and which is incorporated in its entirety by reference herein. It includes a filter element pouch filled with particulate material coated with a flowable, aloe vera extract. The mask also includes a retainer for holding the filter element over the mouth and nose of the wearer. The aloe vera coating has a pH level which is alkaline which aids in the reduction of toxic gases such as hydrogen cyanide, hydrogen chloride and acrolein inhaled by the wearer. The filter element is attached to the retainer and may be provided as a bag-like flexible fabric container adapted to shift so as to conform to the face of the wearer. The filter element itself is adaptable to a variety of different uses for removing smoke particulates and toxic gases from air flowing therethrough.

U.S. Pat. No. 4,520,509, to Ward, discloses a face mask for use in hazardous environments comprising an elastic sheet secured about the lower face and head. The sheet is partially folded to define an open area located forwardly of the wearer's nose and mouth and within which area is removably attached a filter body which may also constitute a countercurrent exchange medium. Fabric closure material on the elastic sheet and filter body serves to removably attach the filter body in place. Additional fabric closure strips of the hook and loop type, when mated to one another, hold the elastic sheet in a partially folded shape to define the aforementioned open area. The filter body may be of a fibrous nature or a molded nose cup shape.

U.S. Pat. No. 4,641,379, to Martin, teaches a face mask comprising a flexible fabric covering the face below the eyes of a wearer and having nose and mouth openings. A flexible skirt affixed to the outer surface of the body portion along a seam which encompasses the nose and mouth openings and also at the tack points forms an air duct which directs expelled air downwardly. Flaps at the nose opening provide a seal around the wearer's nose. An arch affixed to the outer surface of the skirt prevents the mask from resting upon or rubbing against the wearer's nose.

U.S. Pat. No. 5,628,308 to Harges, Jr., et al., shows a heat and fire resistant respiratory filtration mask adapted for protecting firefighters in wildland/brush fire environments. The mask is fabricated of heat and fire resistant material and includes facilitated fasteners for attachment to the head and face of the user. A respiratory filtration pocket accommodates a respiratory filtration element. The pocket has a closable slit for filter removal and replacement. The mask includes openings for exhalation valves of filtration elements and openings for ventilation through the nose and mouth of the wearer.

U.S. Pat. No. 5,322,060, to Johnson, teaches a fire-resistant face mask made of a flexible breathable porous material impregnated with a first solution containing from about 1.8 to about 11.8% by weight ammonium biborate, from about 1.9 to about 6.9% by weight ammonium phosphate, from about 3.7 to about 13.7% by weight ammonium sulfate, from about 0.02 to about 2.0% by weight sodium dodecyl sulfate and from about 92.58 to about 65.6% by weight water; and a second solution containing from about 35% to about 65% by weight lanolin, from about 64.95 to about 30% by weight water and from about 0.05 to about 5.0% by weight lemon fragrance. The mask is breathable and has adsorption capacity for capturing smoke particles, other particulates and for filtering gases.

U.S. Pat. No. 5,058,211, to Hanks, shows a bandana made from an integral piece of fabric folded in a manner to have forward and back panels of substantially triangular shape and an intervening pocket region. The configuration of the article is such that its upper edge is essentially the base of a triangle, and a downwardly directed apex constitutes the lower extremity of the forward panel. Tying ears extend from both extremities of the upper edge. A sheet of compliant thermally insulative material such as a polyurethane sponge is held by sewn seams within the pocket region.

The foregoing patents and prior art devices reflect the current state of the art of which the present inventor is aware. Reference to, and discussion of, these patents is intended to aid in discharging Applicant's acknowledged duty of candor in disclosing information that may be relevant to the examination of prospective claims to the present invention. However, it is respectfully submitted that none of the above-indicated patents disclose, teach, suggest, show, or otherwise render obvious, either singly or when considered in combination, the invention described herein. Specifically, none show or teach or suggest the use of a bandana with elastic edges and hook and loop fastener material for rapid placement and replacement of a fabric pouch containing a particulate filter element which, when worn, is disposed over the nose and mouth of the wearer.

### BRIEF DESCRIPTION OF THE SEVERAL VIEWS OF THE DRAWINGS

The invention will be better understood and objects other than those set forth above will become apparent when consideration is given to the following detailed description thereof. Such description makes reference to the annexed drawing wherein:

FIG. 1 is a front perspective view showing a wearer donning the inventive bandana breathing filter holder of the present invention;

FIG. 2A is a side perspective view thereof;

FIG. 2B is a cross-sectional view thereof; and

FIG. 3 is an exploded perspective view showing the filter mounting elements of the bandana and particulate filter.

### DETAILED DESCRIPTION OF THE INVENTION

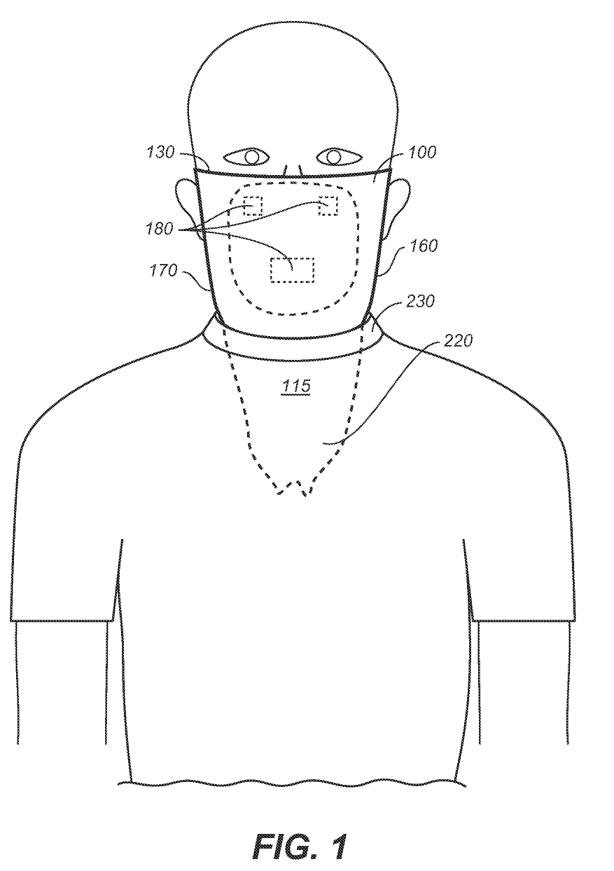

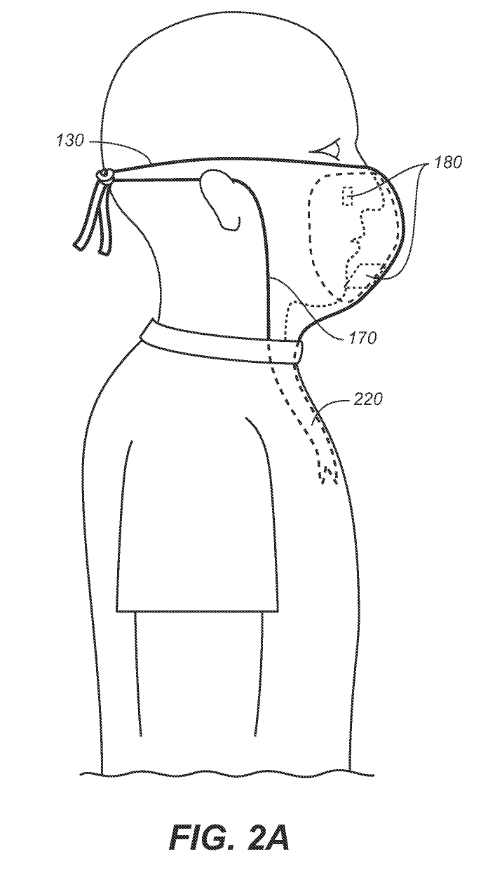

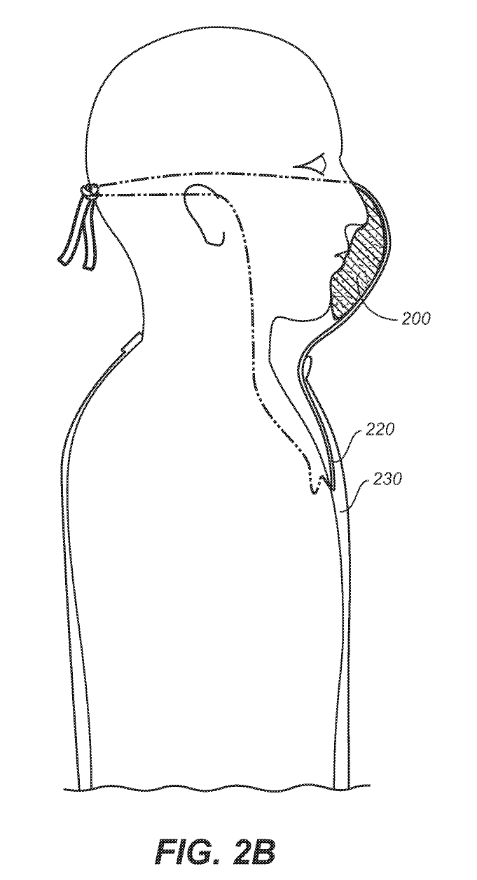

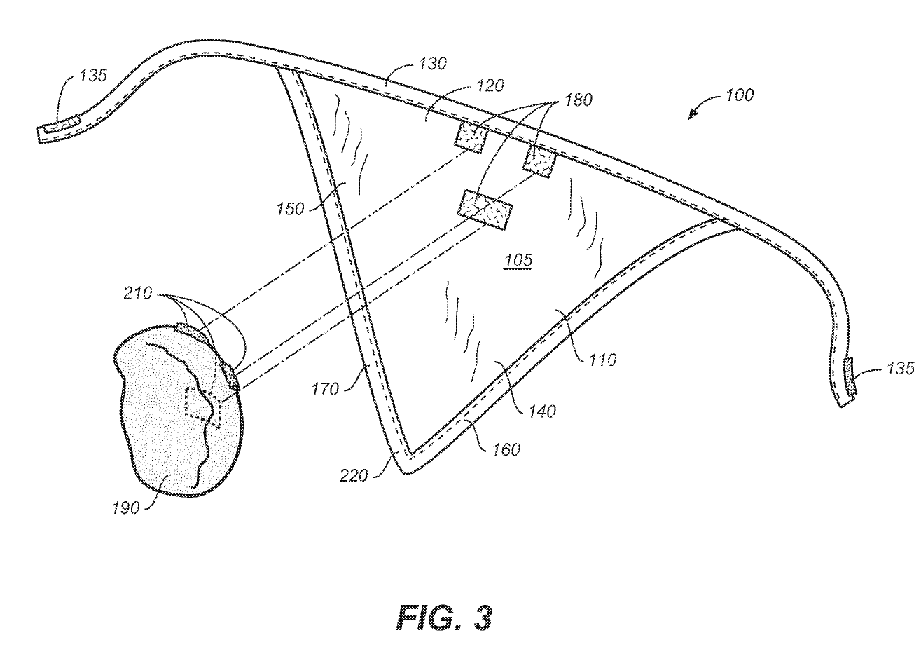

Referring to FIGS. 1 through 3, wherein like reference numerals refer to like components in the various views, there is illustrated therein a new and improved bandana-type holder for a particulate breathing filter, generally denominated 100 herein. These views show that the inventive apparatus comprises a single panel of triangular fabric 110 having an upper (hypotenuse) edge 120 with an elastic strap 130 sewn into the upper edge of the fabric panel. The elastic strap 130 may include hook and loop material 135 at each end and is therefore adapted for either tying or fastening the bandana on the face of the wearer using the portions of the strap extending beyond the junctions of the sides and upper edge of the fabric panel. This is done by draping the straps immediately over the ears of the wearer and then tying or fastening the ends behind and around the head of the wearer.

First and second downwardly depending side edges 140, 150 also include elastic bands 160, 170 sewn into each edge. The fabric is preferably a fire resistant material.

The fabric panel has an inner surface 105 and an outer surface 115. Immediately under the upper edge and disposed on the inner surface are one or more sewn-in panels of hook and loop fastener material 180, or another kind of connection element, which provides attachment means for a particulate filter element 190. The filter element is preferably a pouch 200 filled with particulate smoke and gas filtering material, such as that described in Finch et al, U.S. Pat. No. 6,338,340, incorporated in its entirety by reference herein. The pouch has hook and loop material 210 or other connection means complementary to that on the bandana. Thus, for instance, if the connection element on the bandana were other than hook and loop material, including such elements as buttons or snaps, the complementary button or snap element would be disposed on the pouch.

When a filter element is attached to the bandana and the bandana tied as described above, the filter element is disposed immediately in front of the wearer's nose and mouth and is captured between the bandana fabric and the wearer's face, thereby providing means to filter smoke laden air in emergency circumstances, such as in a burning building or wildfire. The elastic edges help to ensure a relatively tight fit at the sides of the wearer's face, and the general dimensions of the fabric triangle encourage and enable the user to tuck the lower fabric portion 220 and nearby bandana material into his or her shirt 230, thereby improving the barrier to smoke incursion under the sides of the bandana fabric

Preferably, sides of the fabric panel extend the panel material at least six inches as measured directly from the upper edge of the panel to the lowest point on the sides of the panel. This provides ample material for protecting from smoke intrusion below the wearer's mouth. It will also be appreciated from the foregoing description that while a triangular shape for the fabric panel is best suited for confining the sides of the panel to the wearer's face, other geometrical shapes may be employed, including square, rectangular, trapezoidal, and the like. Indeed, in the event that the fabric panel includes a lower edge substantially parallel to the upper edge, the lower edge may include an elastic strap with portions extending beyond the panel for tying around the head or neck of the wearer, much as with a surgical mask. However, while this configuration may have some advantages, the extra time required for making a second knot makes it less than ideal for use as a rapid deployment article.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention.

What is claimed as invention is:

1. A bandana for holding a particulate breathing filter over the mouth and nose of a wearer, comprising:
a fabric panel having an outer surface, an inner surface, an upper edge, and at least first and second sides depending downwardly from said upper edge;
an upper elastic strap affixed to said upper edge and having first and second ends extending beyond the junction of said first and second sides with said upper edge so as to provide means for tying said bandana around the head of the wearer; and
attachment means disposed on said inner surface of said fabric panel for attaching a particulate air filter pouch having attachment apparatus complementary to said attachment means.

2. The apparatus of claim 1, wherein said fabric panel is made from fire resistant material.

3. The apparatus of claim 1, further including an elastic strap affixed to each of said first and second sides.

4. The apparatus of claim 4, wherein the shape of said fabric panel is selected from the group consisting of triangle, square, rectangle, and trapezoid.

5. The apparatus of claim 1, wherein the shape of said fabric panel is selected from the group consisting of triangle, square, rectangle, and trapezoid.

6. The apparatus of claim 1, wherein said attachment means comprises hook and loop material sewn into said fabric panel.

7. The apparatus of claim 1, wherein said first and second ends of said upper elastic strap include hook and loop material for connection to one another.

8. A method for rapidly protecting a person from smoke inhalation danger in the event of fire, comprising the steps of:
(a) providing a breathing filter bandana for holding a particulate breathing filter over the face of the person, wherein the bandana includes a fabric panel having an outer surface, and inner surface, an upper edge, and at least first and second sides depending downwardly from the upper edge, an upper elastic strap affixed to the upper edge and having first and second ends extending beyond the junction of the first and second sides with the upper edge so as to provide means for fastening the bandana around the head of the wearer, and attachment means disposed on the inner surface of the fabric panel for attaching a particulate air filter pouch having attachment apparatus complementary to the attachment means;
(b) providing a particulate air filter pouch having attachment apparatus complementary to the attachment means on the inner surface of the fabric panel;
(c) attaching the particulate air filter pouch to the attachment means;
(d) placing the bandana over the mouth of the person such that the particulate air filter pouch is generally over the mouth and nose of the person; and
(e) draping the first and second ends of the upper elastic strap over the ears of the person and fastening the first and second ends of the upper elastic strap behind the head of the person.

9. The method of claim 8, further including the step of tucking a portion of the bandana into the front collar area of the wearer's shirt.
